(19) United States
(12) Patent Application Publication
(10) Pub. No.: US 2020/0001109 A1
(43) Pub. Date: Jan. 2, 2020

(54) Methods For Treating Hyperbilirubinemia With Stannsoporfin And Phototherapy

(71) Applicant: INFACARE PHARMACEUTICAL CORPORATION, Trevose, PA (US)

(72) Inventors: Dan R. BURNS, Skillman, NJ (US); Simon J. TULLOCH, Gaithersburg, MD (US)

(21) Appl. No.: 16/300,362

(22) PCT Filed: May 12, 2017
(86) PCT No.: PCT/US17/32382

§ 371 Date: Nov. 9, 2018

Related U.S. Application Data
(60) Provisional application No. 62/335,360, filed on May 12, 2016.

(51) Int. Cl. A61N 5/06; A61K 31/555; A61K 9/00; A61P 7/00

(52) U.S. Cl. CPC A61N 5/0621; A61P 7/00; A61K 9/0019; A61K 31/555

(57) ABSTRACT

Methods of treating hyperbilrubinemia or reducing total serum bilirubin levels include initiating phototherapy on an infant while substantially simultaneously administering to the infant a therapeutic amount of stannsoporfin. Various embodiments are directed to treating hyperbilirubinemia and decreasing bilirubin levels.

### CROSS-REFERENCE TO RELATED APPLICATIONS

This application claims priority under 35 U.S.C. § 365 to PCT/US2017/032382, filed May 12, 2017, which claims priority to U.S. Provisional Patent Application No. 62/335,360 filed on May 12, 2016, entitled “METHODS FOR TREATING HYPERBILIRUBINEMIA WITH STANNSOPORFIN AND PHOTOTHERAPY” which is hereby incorporated by reference in its entirety.

### BACKGROUND

Raised bilirubin levels may lead to potentially dangerous conditions, particularly in infants. In some cases, elevated bilirubin levels result from conditions that cause an increase in bilirubin production, while in other conditions the removal of bilirubin is affected. In some instances, it is a combination of an increase in bilirubin production as well as a diminished removal rate of serum bilirubin. Increased bilirubin levels may lead to hyperbilirubinemia and severe hyperbilirubinemia, both of which can be dangerous to a patient.

Hyperbilirubinemia is a common clinical condition seen in both term and pre-term infants. All neonates have some degree of hyperbilirubinemia (total serum bilirubin >2 mg/dL) and most have a benign outcome. Jaundice, a clinical yellowing of the skin and sclera as the result of increased serum bilirubin levels, can be recognized in up to 60% of healthy infants within the first week of life and peaks between 96-120 hours of age. Unconjugated bilirubin penetrates the blood brain barrier and is a known central nervous system toxin that injures glial cells and causes a typical inflammatory response. With increasing levels of bilirubin, central nervous system dysfunction begins to appear. Acute bilirubin encephalopathy or bilirubin induced neurologic dysfunction (BIND) is a clinical syndrome consisting of alterations in muscle tone fluctuating from hypotonia to marked hypertonia, varying degrees of impaired alertness, decreased feeding and irritability. If left untreated, the encephalopathy progresses to a permanent central nervous system injury with bilirubin staining of brain nuclei known as kernicterus. Clinically, infants with kernicterus present with motor impairment manifest as cerebral palsy, ataxia, mental retardation, and hearing loss. Although kernicterus is rare, it is a devastating, but preventable, disorder with lifelong consequences for the infant and family.

Guidelines for initiating phototherapy (PT) and exchange transfusion (ET) in infants with jaundice/hyperbilirubinemia have been published by the American Academy of Pediatrics (see FIGS. 1 and 2). (American Academy of Pediatrics Subcommittee on Hyperbilirubinemia: (Pediatrics 2004; 114:297-316.)). Implementation of these guidelines, combined with bilirubin screening, allow for the early identification and treatment of infants with hyperbilirubinemia. Despite this, neonatal jaundice remains the most common cause of hospital readmission for term and near-term infants.

The management of neonatal jaundice poses a significant healthcare burden. The current treatment options for neonates at risk for severe hyperbilirubinemia are insufficient. Phototherapy, the current standard of care, works by enhancing the excretion of bilirubin but does not inhibit its production. Phototherapy is generally an effective therapy for lowering bilirubin levels and many infants respond to treatment over time. However, there are occasions when, despite phototherapy, bilirubin will continue to rise necessitating extended periods of phototherapy, and rarely, to levels where exchange transfusion is necessary. More commonly, hospital readmission for treatment of hyperbilirubinemia is required after the infant has been discharged. Practitioners view phototherapy as safe because the treatment guidelines are evidence-based and were developed based on a review of the phototherapy experience in millions of neonates over decades. However, no well controlled registration grade studies have been conducted and little in the way of long-term outcome studies on the use of high intensity (>30 μW) phototherapy as recommended by the AAP guidelines. Short-term side effects of phototherapy have been noted in the literature, including loose watery stools, an increase in insensible water loss, skin rashes and retinal damage. Opaque eye patches are used to protect the patient's eyes during phototherapy to prevent possible retinal damage. Exchange transfusion is a last resort treatment that has an associated mortality and morbidity.

Accordingly, more and different treatments for reducing bilirubin production, increasing bilirubin excretion, or both, are desirable as are other methods of treating hyperbilirubinemia, increased bilirubin production, and/or reducing total serum bilirubin levels.

### SUMMARY

In some embodiments, a method of decreasing bilirubin levels in an infant comprising placing an infant on phototherapy and substantially simultaneously administering to the infant a therapeutic amount of stannsoporfin is provided. In some embodiments, a method of treating hyperbilirubinemia in an infant comprising placing an infant on phototherapy and substantially simultaneously administering to the infant a therapeutic amount of stannsoporfin is provided.

Some embodiments provide a method of decreasing bilirubin levels in an infant in need thereof, the method comprising initiating phototherapy in the infant and substantially simultaneously with the initiation of phototherapy, administering to the infant a therapeutic amount of stannsoporfin.

In some embodiments, the administering of the therapeutic amount of stannsoporfin occurs no more than 60 minutes apart from the initiation of phototherapy.

In some embodiments, the administering of the therapeutic amount of stannsoporfin occurs no more than 30 minutes apart from the initiation of phototherapy.

In some embodiments, the administering of the therapeutic amount of stannsoporfin occurs no more than 60 minutes before the initiation of phototherapy.

In some embodiments, the administering of the therapeutic amount of stannsoporfin occurs no more than 30 minutes before the initiation of phototherapy.

In some embodiments, the administering of the therapeutic amount of stannsoporfin occurs no more than 60 minutes after the initiation of the phototherapy.

In some embodiments, the administering of the therapeutic amount of stannsoporfin occurs no more than 30 minutes after the initiation of the phototherapy.

In some embodiments, the therapeutic amount of stannsoporfin is selected from the group consisting of 1.5 mg/kg, 3.0 mg/kg and 4.5 mg/kg.

In some embodiments, prior to initiating phototherapy, the infant has a total serum bilirubin level at or above the age-specific threshold for initiating phototherapy per the AAP guidelines.

In some embodiments, phototherapy is stopped when the total serum bilirubin level crosses below the age-specific threshold for initiating phototherapy per the AAP guidelines.

In some embodiments, the infant is direct antiglobulin test (DAT) negative with a reticulocyte count greater than 6%.

In some embodiments, prior to initiating phototherapy, the infant is about twenty-four hours old or less.

In some embodiments, prior to initiating phototherapy, the infant has a total serum bilirubin level 1 mg/dL or more below the threshold for phototherapy initiation of the AAP guidelines.

In some embodiments, prior to initiating phototherapy, the infant has a total serum bilirubin level 2 mg/dL or more below the threshold for phototherapy initiation of the AAP guidelines.

In some embodiments, prior to initiating phototherapy, the infant has a total serum bilirubin level 3 mg/dL or more below the threshold for phototherapy initiation of the AAP guidelines.

In some embodiments, the infant is at increased risk for hyperbilirubinemia.

In some embodiments, the increased risk is due to hemolytic disease, ABO incompatibility, Rh incompatibility, or G6PD deficiency.

In some embodiments, the increased risk is measured as a rate of rise of bilirubin greater or equal to 0.2 mg/dL/hour.

Some embodiments provide a method of treating hyperbilirubinemia in an infant in need thereof, the method comprising initiating phototherapy in the infant and substantially simultaneously with the initiation of phototherapy, administering to the infant a therapeutic amount of stannsoporfin.

In some embodiments, the administering of the therapeutic amount of stannsoporfing occurs no more than 60 minutes apart from the initiation of phototherapy.

In some embodiments, the administering of the therapeutic amount of stannsoporfin occurs no more than 30 minutes apart from the initiation of phototherapy.

In some embodiments, the administering of the therapeutic amount of stannsoporfin occurs no more than 60 minutes before the initiation of phototherapy.

In some embodiments, the administering of the therapeutic amount of stannsoporfin occurs no more than 30 minutes before the initiation of phototherapy.

In some embodiments, the administering of the therapeutic amount of stannsoporfin occurs no more than 60 minutes after the initiation of the phototherapy.

In some embodiments, the administering of the therapeutic amount of stannsoporfin occurs no more than 30 minutes after the initiation of the phototherapy.

In some embodiments, the therapeutic amount of stannsoporfin is selected from the group consisting of 1.5 mg/kg, 3.0 mg/kg and 4.5 mg/kg.

In some embodiments, prior to imitating phototherapy, the infant has a total serum bilirubin level at or above the age-specific threshold for initiating phototherapy per the AAP guidelines.

In some embodiments, phototherapy is stopped when the total serum bilirubin level crosses below the age-specific threshold for initiating phototherapy per the AAP guidelines.

In some embodiments, the infant is direct antiglobulin test (DAT) negative with a reticulocyte count greater than 6%.

In some embodiments, prior to initiating phototherapy, the infant is 24 hours old or less.

In some embodiments, wherein prior to initiating phototherapy, the infant has a total serum bilirubin level 1 mg/dL or more below the threshold for phototherapy initiation of the AAP guidelines.

In some embodiments, prior to initiating phototherapy, the infant has a total serum bilirubin level 2 mg/dL or more below the threshold for phototherapy initiation of the AAP guidelines.

In some embodiments, prior to initiating phototherapy, the infant has a total serum bilirubin level 3 mg/dL or more below the threshold for phototherapy initiation of the AAP guidelines.

### BRIEF DESCRIPTION OF THE FIGURES

FIG. 1 is a graph showing the thresholds for instituting phototherapy in hospitalized infants of 35 or more weeks gestation as established by the American Academy of Pediatrics Subcommittee on Hyperbilirubinemia (Pediatrics 2004; 114:297-316.)

FIG. 2 is a graph showing the thresholds for instituting exchange transfusion in hospitalized infants of 35 or more weeks gestation as established by American Academy of Pediatrics Subcommittee on Hyperbilirubinemia (Pediatrics 2004; 114:297-316.)

### DETAILED DESCRIPTION

It should be noted that as used herein and in the appended claims, the singular forms “a”, “an”, and “the” include plural reference unless the context clearly dictates otherwise. Thus, for example, reference to “a compound” is a reference to one or more compounds and equivalents thereof known to those skilled in the art, and so forth.

As used herein, the term “about” means plus or minus 10% of the numerical value of the number with which it is being used unless otherwise indicated or custom in the art dictates otherwise. Therefore, “about 50%” means in the range of 45%-55%.

“Administering” when used in conjunction with a therapeutic means to apply, inject, or otherwise provide, a therapeutic directly into or onto a target tissue or to apply, inject, or otherwise provide a therapeutic to a patient systemically. Thus, as used herein, the term “administering”, when used in conjunction with stannsoporfin, can include, but is not limited to, providing the stannsoporfin into or onto the target tissue; providing the stannsoporfin systemically to a patient by, e.g., injection (e.g. intravenous, intramuscular, or sub-cutaneous) whereby the therapeutic reaches the target tissue. “Administering” a composition may be accomplished by injection (intravenous, intramuscular, or subcutaneous), topical administration, oral, or by other method, alone in combination with other known techniques.

The term “animal,” “subject” or “patient” as used herein includes, but is not limited to, humans and non-human vertebrates such as wild, domestic and farm animals. Most preferably, “animal,” “subject,” or “patient” refers to humans, particularly infants.

The term “improves” is used to convey generally positive changes in the appearance, form, characteristics and/or the physical attributes of the subject or tissue to which treatment is being provided, applied or administered. The change may be demonstrated by, for example and not limitation, any of the following, alone or in combination: enhanced appearance of the skin; reduced need for exchange transfusion; reduced need for or duration of phototherapy; decrease in bilirubin levels; decrease in rebound (e.g. decrease in the likelihood of restarting phototherapy after being off of phototherapy for 6 hours of more); decrease in jaundice; prevention or reduction of zone 5 jaundice; decrease in incidence of, or need for, intravenous immunoglobulin administration; reduction in the length of hospital stay (compared to phototherapy alone); decrease in rate or likelihood of hospital readmission, etc.

The term “inhibiting” includes reducing the likelihood of the onset of symptoms, alleviating symptoms, or eliminating the disease, condition or disorder.

By “pharmaceutically acceptable,” it is meant the item described, e.g. composition, carrier, diluent, excipient, etc. is compatible with the other ingredients of the formulation and not deleterious to the recipient thereof. That is, although some negative or undesirable effects may be expected and tolerable, the pharmaceutically acceptable item is accepted for use by the US FDA, particularly in combination with the other ingredients of the formulation.

As used herein, the term “therapeutic” means an agent used to treat, combat, ameliorate, inhibit, or improve an unwanted condition, disorder or disease, or symptom thereof, of a patient. In part, embodiments described herein are directed to the treatment of hyperbilirubinemia and/or the reduction in total serum bilirubin.

A “therapeutic amount” or “effective amount” of a composition is a predetermined amount calculated to achieve the desired effect, i.e., to treat, prevent or reduce jaundice or hyperbilirubinemia; to reduce bilirubin production; to increase bilirubin excretion; or combination thereof; or to reduce total serum bilirubin and/or total cutaneous bilirubin; or to otherwise delay, inhibit, or slow the progression of hyperbilirubinemia; to enhance the appearance of the skin; to reduce need for exchange transfusion; to reduce need for, or duration of, phototherapy; to decrease bilirubin levels; to decrease rebound (e.g. decrease in the likelihood of restarting phototherapy after being off of phototherapy for 6 hours of more); to decrease jaundice; to prevent or reduct zone 5 jaundice; to decrease the incidence of, or need for intravenous immunoglobulin administration; to reduce the length of hospital stay; to decrease the rate or likelihood of hospital readmission, etc. The activity contemplated by the methods disclosed herein includes both therapeutic and/or prophylactic treatment, as appropriate. The specific dose of stannsoporfing administered according to the methods disclosed herein to obtain therapeutic and/or prophylactic effects will, of course, be determined by the particular circumstances surrounding the case, including, for example, the route of administration, and the condition being treated. Stannsoporfin is effective in various dosages. However, it will be understood that the effective amount administered will be determined by the physician in light of the relevant circumstances including the condition to be treated, the chosen route of administration, and other factors, and therefore the dosage ranges disclosed herein are exemplary only. A therapeutic amount of stannsoporfin as disclosed herein is typically an amount such that when it is administered in a pharmaceutically acceptable composition, it is sufficient to achieve an effective systemic concentration or local concentration in the tissue.

The terms “treat,” “treated,” or “treating” as used herein refer to both therapeutic measures and prophylactic or preventative measures, wherein the object is to prevent or slow down (lessen) an undesired physiological condition, disorder or disease, or symptom thereof, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease or symptoms thereof; delay in onset or slowing of the progression of the condition, disorder or disease or symptoms thereof; amelioration of the condition, disorder or disease state or symptom thereof; and remission (whether partial or total), whether detectable or undetectable, or enhancement or improvement of the condition, disorder or disease or symptom thereof. Treatment includes eliciting a clinically significant response without excessive levels of side effects. Treatment also includes prolonging survival as compared to expected survival if not receiving treatment.

As used herein, the term “baseline” refers to the patient's total serum bilirubin levels prior to administration of therapeutic treatment or prophylactic or preventative measures. In some embodiments, an infant's baseline serum bilirubin levels serves as the foundation against which to measure changes in the patient's serum bilirubin levels.

In some embodiments, the infant in need of treatment may have one or more risk factors for hyperbilirubinemia, such as but not limited to ABO incompatibility, Rh incompatibility, G6PD deficiency, hemolytic disease, DAT positive, DAT negative with reticulocyte count greater than 6%, etc.

Bilirubin is formed from the degradation of the heme component of hemoglobin. Infants with isoimmune hemolytic disease, such as ABO incompatibility or Rh incompatibility, are at increased risk for severe hyperbilirubinemia due to an increased rate of red cell destruction and, thus, an increase in bilirubin production. Infants born with G6PD deficiency are also at increased risk of hemolysis and severe hyperbilirubinemia during the neonatal period. Since newborn infants have relatively immature liver function, they do not conjugate bilirubin well, which results in accumulation of unconjugated bilirubin. Thus, for infants with hemolytic disease, bilirubin levels may rise rapidly and intervention may be required in the first 24-72 hours of life. At present, phototherapy using blue light (430-490 nm) is the most frequently used treatment for hyperbilirubinemia. The blue light employed in phototherapy systems converts unconjugated bilirubin to less toxic water soluble photoisomers that can be excreted. Thus, phototherapy enhances the excretion of bilirubin but has no impact on the production of bilirubin.

Since production of bilirubin is significantly increased in infants with hemolytic disease, bilirubin levels may continue to increase despite phototherapy. Infants who do not respond to phototherapy are treated by exchange transfusion; however, exchange transfusion is considered a therapy of last resort because of associated morbidity and mortality. Guidelines for initiating phototherapy and exchange transfusion in infants with jaundice/hyperbilirubinemia have been published and are widely accepted.

Stannsoporfin is a heme oxygenase inhibitor that acts to reduce bilirubin production.

Infants with jaundice due to hemolytic disease, sometimes secondary to ABO or Rh incompatibility or glucose-6-phosphate dehydrogenase (G6PD) deficiency have elevated production of bilirubin. Phototherapy may be less effective in this population than in infants without hemolysis, as it does not alter the rate of bilirubin production. In addition, phototherapy has not eliminated the need for exchange transfusion. Therefore, there is a medical need to improve treatment of hyperbilirubinemia in infants with hemolytic disease from any etiology.

Of note, G6PD deficiency is the most common enzyme deficiency worldwide, and is associated with a spectrum of diseases including neonatal hyperbilirubinemia. This X-linked inherited disorder most commonly affects persons of African, Asian, Mediterranean, or Middle-Eastern descent. However, immigration and intermarriage has made G6PD deficiency a global problem. Approximately 400 million people are affected worldwide. G6PD deficiency should be considered in neonates who develop hyperbilirubinemia within the first 24 hours of life, have a history of jaundice in a sibling, bilirubin levels greater than the 95th percentile, and in Asian males. G6PD deficiency has also been listed as a risk factor of severe hyperbilirubinemia by the American Academy of Pediatrics.

ABO incompatibility occurs in 20% to 25% of pregnancies, and a proportion of these develop problematic hyperbilirubinemia due to hemolysis. The direct antiglobulin test (DAT) or Coombs test may be used to help diagnose hemolytic disease of the newborn (HDN) due to an incompatibility between the blood types of a mother and baby. If the DAT is positive, then there are anti red cell antibodies present. If the DAT is negative, then antibodies are not detectable and the jaundice may be due to some other cause. A recent survey of published studies shows that in cases of hemolytic anemia, a negative DAT has been reported in 3-11% of the cases considered. They describe three possible reasons for the absence of a positive result: IgG sensitization below the threshold of detection for the chosen antiglobulin reagent, possible removal of low-affinity IgG, or red cell sensitization by IgA or IgM alone and without associated complement fixation.

Heme oxygenase is the enzyme involved in the rate-limiting step of the catabolism of heme to bilirubin, catalyzing the transformation of heme to biliverdin and subsequent conversion by biliverdin reductase to bilirubin. Stannsoporfin is a competitive inhibitor of heme oxygenase and, as such, temporarily blocks the production of bilirubin from heme. Since infants with hyperbilirubinemia with hemolytic conditions such as G6PD deficiency or ABO/Rh incompatibility are likely to be high producers of bilirubin, they are the ideal population for the study of stannsoporfin, which inhibits production of bilirubin.

In certain embodiments, a method of decreasing bilirubin levels in an infant comprises placing an infant on phototherapy and substantially simultaneously administering to the infant a therapeutic amount of stannsoporfin.

The phrase “placing an infant on phototherapy” means initiating phototherapy on an infant. Thus, “substantially simultaneous” administration of the stansoporfin, typically given as a single dose, is measured relative to the initiation of the phototherapy.

In embodiments described herein, the method of decreasing bilirubin levels in an infant comprising placing an infant on phototherapy and substantially simultaneously administering to the infant a therapeutic amount of stannsoporfin decreases the level of bilirubin. In embodiments described herein, the method of decreasing bilirubin levels in an infant comprising placing an infant on phototherapy and substantially simultaneously administering to the infant a therapeutic amount of stannsoporfin decreases the need for phototherapy. In embodiments described herein, the method of decreasing bilirubin levels in an infant comprising placing an infant on phototherapy and substantially simultaneously administering to the infant a therapeutic amount of stannsoporfin decreases the duration of phototherapy. In embodiments described herein, the method of decreasing bilirubin levels in an infant comprising placing an infant on phototherapy and substantially simultaneously administering to the infant a therapeutic amount of stannsoporfin eliminates or decreases the need for exchange therapy.

In some embodiments, a method of treating hyperbilirubinemia in an infant comprises placing an infant on phototherapy and substantially simultaneously administering to the infant a therapeutic amount of stannsoporfin.

In embodiments described herein, the method of treating hyperbilirubinemia in an infant comprising placing an infant on phototherapy and substantially simultaneously administering to the infant a therapeutic amount of stannsoporfin decreases the level of bilirubin. In embodiments described herein, the method of treating hyperbilirubinemia in an infant comprising placing an infant on phototherapy and substantially simultaneously administering to the infant a therapeutic amount of stannsoporfin decreases the need for phototherapy. In embodiments described herein, the method of treating hyperbilirubinemia in an infant comprising placing an infant on phototherapy and substantially simultaneously administering to the infant a therapeutic amount of stannsoporfin decreases the duration of phototherapy. In embodiments described herein, the method of treating hyperbilirubinemia in an infant comprising placing an infant on phototherapy and substantially simultaneously administering to the infant a therapeutic amount of stannsoporfin eliminates or decreases the need for exchange therapy.

In embodiments described herein, the placing of the infant on phototherapy occurs within 60 minutes or within 30 minutes before or after administering of the therapeutic amount of stannsoporfin. In embodiments described herein, the placing of the infant in phototherapy occurs immediately prior to, during, or immediately after the administration of the therapeutic amount of stannsoporfin.

In some embodiments, the stansoporfin is administered up to 60 minutes before initiating phototherapy. In some embodiments, the stansoporfin is administered up to 30 minutes before initiating phototherapy.

In some embodiments, the stansoporfin is administered up to 60 minutes after initiating phototherapy. In some embodiments, the stansoporfin is administered up to 30 after before initiating phototherapy.

In embodiments described herein, the infant is term or near-term. In embodiments described herein, the infant's gestational age is greater than or equal to 35 weeks or less than or equal to 43 weeks.

In embodiments described herein, the therapeutic amount of stannsoporfin is between about 1.5 mg/kg and about 100 mg/kg, based on birth weight of the infant. In embodiments described herein, the therapeutic amount of stannsoporfin is between about 3.0 mg/kg and about 75 mg/kg. In embodiments described herein, the therapeutic amount of stannsoporfin is between about 4.5 mg/kg and about 50 mg/kg. In embodiments described herein, the therapeutic amount of stannsoporfin is between about 1.5 mg/kg and about 10 mg/kg. In embodiments described herein, the therapeutic amount of stannsoporfin is about 1.5 mg/kg to about 4.5 mg/kg. In embodiments described herein, the therapeutic amount of stannsoporfin is about 1.5 mg/kg. In embodiments described herein, the therapeutic amount of stannsoporfin is about 3.0 mg/kg. In embodiments described herein, the therapeutic amount of stannsoporfin is about 4.5 mg/kg.

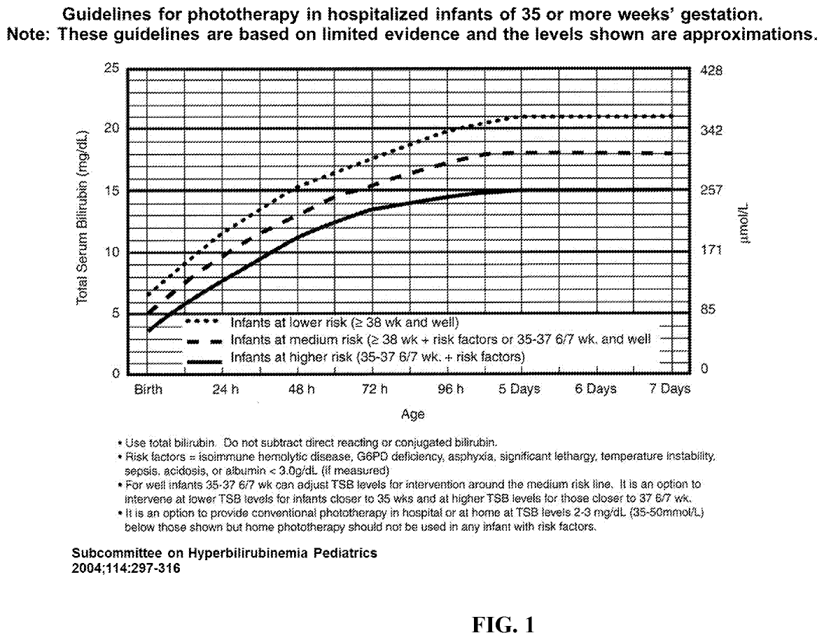

In embodiments described herein, the infant, prior to treatment, has a total serum bilirubin (TSB) at or above the age-specific threshold for initiating phototherapy per the American Academy of Pediatrics (AAP) guidelines (Tables 1, 2, 3 and FIG. 1). In embodiments described herein, the infant requires the initiation of phototherapy. In embodiments described herein, the infant, prior to treatment, has a total serum bilirubin (TSB) within 1 mg/dL of the American Academy of Pediatrics (AAP) guidelines for phototherapy initiation, within 2 mg/dL of the American Academy of Pediatrics (AAP) guidelines for phototherapy initiation and within 3 mg/dL of the American Academy of Pediatrics (AAP) guidelines for phototherapy initiation. In embodiments described herein, the infant has a TSB selected from the group consisting of at or below 1 mg/dL, at or below 2 mg/dL, and at or below 3 mg/dL of the phototherapy threshold.

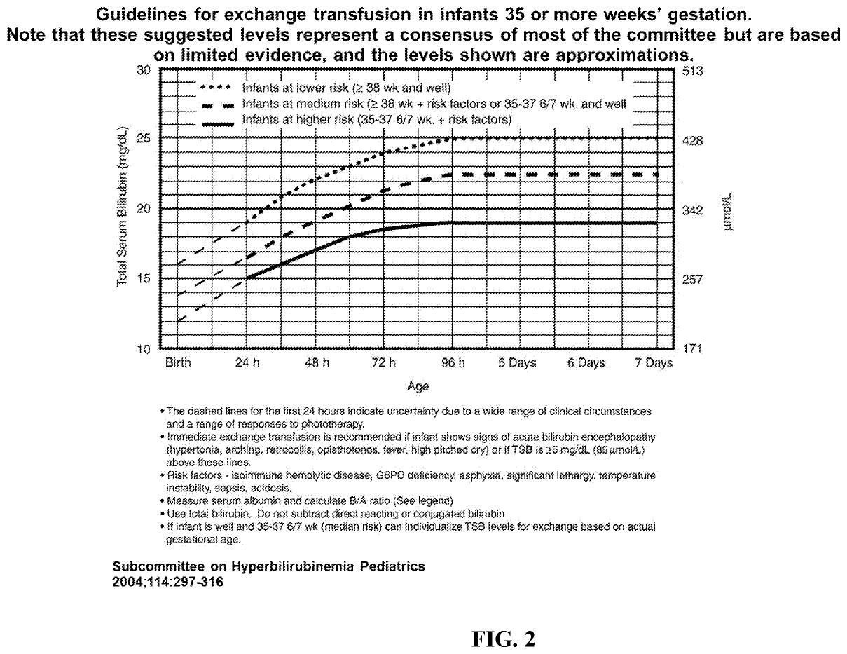

In embodiments described herein, the infant, prior to treatment, has a total serum bilirubin (TSB) at or above the age-specific threshold for initiating exchange transfusion per the American Academy of Pediatrics (AAP) guidelines (FIG. 2). In some embodiments described herein, the infant, prior to treatment, has a total serum bilirubin (TSB) within 1 mg/dL of the American Academy of Pediatrics (AAP) guidelines for initiating exchange transfusion, within 2 mg/dL of the American Academy of Pediatrics (AAP) guidelines for initiating exchange transfusion and within 3 mg/dL of the American Academy of Pediatrics (AAP) guidelines for initiating exchange transfusion. In embodiments described herein, the infant has a TSB selected from the group consisting of at or below 1 mg/dL, at or below 2 mg/dL, and at or below 3 mg/dL the threshold for initiating exchange transfusion.

In embodiments described herein, the need for treatment is initiated when the infant is at an increased risk for hyperbilirubinemia. In embodiments described herein, the infant has an increased risk of hyperbilirubinemia, wherein the rate of rise of bilirubin is greater than or equal to 0.2 mg/dL/hour. In embodiments described herein, the infant has hemolytic disease. In embodiments described herein, the infant has ABO incompatibility. In embodiments described herein, the infant has Rh incompatibility. In embodiments described herein, the infant has G6PD deficiency. In embodiments described herein, the infant is between 0 to 48 hours old, between 0 to 36 hours old, and between 0 and 24 hours old and between 0 and 12 hours old. In embodiments described herein, the infant is less than 72 hours old, less than 48 hours old, less than 36 hours old, less than 24 hours old and less than 12 hours old. In embodiments described herein, the infant is 24 hours old or less.

In embodiments described herein, the infant is between 0 to 48 hours old, is ABO incompatible, and is DAT-positive. In embodiments described herein, the infant is between 0 to 48 hours old, is Rh incompatible, and is DAT-positive. In embodiments described herein, the infant is age 0 to 72 hours old, and is G6PD deficient. In embodiments described herein, the infant is age 0 to 48 hours, is ABO incompatible, is DAT negative, and has an increased reticulocyte count (>6%). In embodiments described herein, the infant is between 0 to 48 hours old, is Rh incompatible, is DAT negative, and has an increased reticulocyte count (>6%).

The embodiments disclosed herein illustrating the method and materials used may be further understood by reference to the following non-limiting examples.

### Examples

A Phase 2b multicenter, double-blind, randomized, placebo-controlled parallel group safety and efficacy trial of two doses of stannsoporfin administered as a single intramuscular injection in combination with phototherapy in term and near-term infants has been proposed and performed.

Infants will be randomized to one of three treatment arms: placebo, 3.0 mg/kg, or 4.5 mg/kg of stannsoporfin in a 1:1:1 ratio. Infants aged 0 to 48 hours (at the time the qualifying TSB is drawn) with ABO or Rh incompatibility who are DAT-positive, or age 0 to 72 hours (at the time the qualifying TSB is drawn) with G6PD deficiency, confirmed by a documented blood test, who require the initiation of phototherapy are eligible for the study. In addition, patients aged 0 to 48 hours (at the time the qualifying TSB is drawn) with ABO or Rh incompatibility who are DAT negative (or status unknown), and have an increased reticulocyte count (>6%), and require the initiation of phototherapy are eligible for the study.

Patients must meet the following inclusion criteria and have none of the exclusion criteria to be eligible for inclusion in the study.

Inclusion Criteria: 1) Term and near-term infants ≥35 and ≤43 weeks gestational age (GA), age 0-48 hours (at the time the qualifying TSB is drawn) with ABO or Rh incompatibility (anti C, c, D, E or e) who are DAT-positive, or age 0-72 hours (at the time the qualifying TSB is drawn) with G6PD deficiency as confirmed by a documented blood test; OR 2) Term and near-term infants ≥35 and ≤43 weeks GA, age 0-48 hours (at the time the qualifying TSB is drawn) with ABO or Rh incompatibility (anti C, c, D, E or e) who are DAT-negative (or status unknown) and have an increased reticulocyte count (>6%); parental or guardian written consent; birth weight ≥2500 grams; TSB at or above the age-specific threshold for initiating phototherapy per the AAP guidelines (see FIG. 1 and Tables 1, 2 and 3); and parents agree to observe light precautions for 10 days post treatment

TABLE 1
TSB Levels for Screening, Low risk neonates
Low-risk neonates

| Age | Entry criteria | | Threshold PT |
|---|---|---|---|
| (hours)^{a} | (mg/dL) | | (mg/dL) |
| 0 | Within | 4.6 | 6.6 |
| 1 | 2 mg/dL | 4.8 | 6.8 |
| 2 | of PT | 5.0 | 7.0 |
| 3 | threshold | 5.2 | 7.2 |
| 4 | | 5.4 | 7.4 |
| 5 | | 5.6 | 7.6 |
| 6 | | 5.8 | 7.8 |
| 7 | | 6.0 | 8.0 |
| 8 | | 6.2 | 8.2 |
| 9 | | 6.5 | 8.5 |
| 10 | | 6.7 | 8.7 |
| 11 | | 6.9 | 8.9 |
| 12 | Within | 6.1 | 9.1 |
| 13 | 3 mg/dL | 6.3 | 9.3 |
| 14 | PT of | 6.5 | 9.5 |
| 15 | threshold | 6.7 | 9.7 |
| 16 | | 6.9 | 9.9 |
| 17 | | 7.1 | 10.1 |
| 18 | | 7.3 | 10.4 |
| 19 | | 7.5 | 10.6 |
| 20 | | 7.8 | 10.8 |
| 21 | | 8.0 | 11.1 |
| 22 | | 8.2 | 11.3 |
| 23 | | 8.5 | 11.5 |
| 24 | | 8.7 | 11.7 |
| 25 | | 8.9 | 11.9 |
| 26 | | 9.0 | 12.0 |
| 27 | | 9.2 | 12.2 |
| 28 | | 9.3 | 12.3 |
| 29 | | 9.5 | 12.5 |
| 30 | | 9.6 | 12.7 |
| 31 | | 9.8 | 12.8 |
| 32 | | 9.9 | 13.0 |
| 33 | | 10.1 | 13.1 |
| 34 | | 10.2 | 13.3 |
| 35 | | 10.4 | 13.4 |
| 36 | | 10.5 | 13.6 |
| 37 | | 10.7 | 13.7 |
| 38 | | 10.8 | 13.9 |
| 39 | | 11.0 | 14.0 |
| 40 | | 11.2 | 14.2 |
| 41 | | 11.3 | 14.3 |
| 42 | | 11.5 | 14.5 |
| 43 | | 11.6 | 14.6 |
| 44 | | 11.7 | 14.7 |
| 45 | | 11.9 | 14.9 |
| 46 | | 12.0 | 15.0 |
| 47 | | 12.1 | 15.2 |
| 48 | | 12.2 | 15.3 |

^{a}Age in hours should represent the subject's actual age, i.e., 10.75, which corresponds to the age 10 row (10 hours and 0 minutes to 10 hours and 59 minutes)

TABLE 2
TSB Levels for Screening, Medium risk neonates
Medium Risk Neonates

| Age | Enrollment | | Threshold |
|---|---|---|---|
| (hours) | criteria (mg/dl) | | PT (mg/dl) |
| 0 | Within | 3 | 5 |
| 1 | 2 mg/dL | 3.2 | 5.2 |
| 2 | below PT | 3.4 | 5.4 |
| 3 | threshold | 3.7 | 5.7 |
| 4 | | 3.9 | 5.9 |
| 5 | | 4.1 | 6.1 |
| 6 | | 4.3 | 6.3 |
| 7 | | 4.5 | 6.5 |
| 8 | | 4.7 | 6.7 |
| 9 | | 5 | 7 |
| 10 | | 5.2 | 7.2 |
| 11 | | 5.4 | 7.4 |
| 12 | Within | 4.6 | 7.6 |
| 13 | 3 mg/dL | 4.8 | 7.8 |
| 14 | below PT | 4.9 | 7.9 |
| 15 | threshold | 5.1 | 8.1 |
| 16 | | 5.3 | 8.3 |
| 17 | | 5.4 | 8.4 |
| 18 | | 5.8 | 8.8 |
| 19 | | 6 | 9.0 |
| 20 | | 6.2 | 9.2 |
| 21 | | 6.4 | 9.4 |
| 22 | | 6.5 | 9.5 |
| 23 | | 6.7 | 9.7 |
| 24 | | 6.9 | 9.9 |
| 25 | | 7.1 | 10.1 |
| 26 | | 7.2 | 10.2 |
| 27 | | 7.4 | 10.4 |
| 28 | | 7.5 | 10.5 |
| 29 | | 7.7 | 10.7 |
| 30 | | 7.8 | 10.8 |
| 31 | | 8 | 11.0 |
| 32 | | 8.1 | 11.1 |
| 33 | | 8.3 | 11.3 |
| 34 | | 8.4 | 11.4 |
| 35 | | 8.6 | 11.6 |
| 36 | | 8.7 | 11.7 |
| 37 | | 8.8 | 11.8 |
| 38 | | 8.9 | 11.9 |
| 39 | | 9.1 | 12.1 |
| 40 | | 9.2 | 12.2 |
| 41 | | 9.3 | 12.3 |
| 42 | | 9.4 | 12.4 |
| 43 | | 9.5 | 12.5 |
| 44 | | 9.6 | 12.6 |
| 45 | | 9.8 | 12.8 |
| 46 | | 9.9 | 12.9 |
| 47 | | 10 | 13.0 |
| 48 | | 10.1 | 13.1 |
| 49 | | 10.2 | 13.2 |
| 50 | | 10.4 | 13.4 |
| 51 | | 10.5 | 13.5 |
| 52 | | 10.6 | 13.6 |
| 53 | | 10.7 | 13.7 |
| 54 | | 10.9 | 13.9 |
| 55 | | 11 | 14.0 |
| 56 | | 11.1 | 14.1 |
| 57 | | 11.2 | 14.2 |
| 58 | | 11.4 | 14.4 |
| 59 | | 11.5 | 14.5 |
| 60 | | 11.6 | 14.6 |
| 61 | | 11.7 | 14.7 |
| 62 | | 11.8 | 14.8 |
| 63 | | 11.8 | 14.8 |
| 64 | | 11.9 | 14.9 |
| 65 | | 12 | 15.0 |
| 66 | | 12.1 | 15.1 |
| 67 | | 12.1 | 15.1 |
| 68 | | 12.2 | 15.2 |
| 69 | | 12.3 | 15.3 |
| 70 | | 12.4 | 15.4 |
| 71 | | 12.4 | 15.4 |
| 72 | | 12.5 | 15.5 |

a: Age in hours should represent the patient's actual age, i.e., 10.75, which corresponds to the age 10 row (10 hours and 0 minutes to 10 hours and 59 minutes)

TABLE 3
TSB Levels for Screening, High risk neonates
High Risk Neonates

| Age | Enrollment | | Threshold |
|---|---|---|---|
| (hours) | criteria (mg/dl) | | PT (mg/dl) |
| 0 | Within | 1.6 | 3.6 |
| 1 | 2 mg/dL | 1.8 | 3.8 |
| 2 | below PT | 2 | 4 |
| 3 | threshold | 2.2 | 4.2 |
| 4 | | 2.4 | 4.4 |
| 5 | | 2.6 | 4.6 |
| 6 | | 2.8 | 4.8 |
| 7 | | 3 | 5 |
| 8 | | 3.2 | 5.2 |
| 9 | | 3.4 | 5.4 |
| 10 | | 3.5 | 5.5 |
| 11 | | 3.7 | 5.7 |
| 12 | Within | 2.9 | 5.9 |
| 13 | 3 mg/dL | 3.1 | 6.1 |
| 14 | below PT | 3.2 | 6.2 |
| 15 | threshold | 3.4 | 6.4 |
| 16 | | 3.5 | 6.5 |
| 17 | | 3.7 | 6.7 |
| 18 | | 4 | 7.0 |
| 19 | | 4.2 | 7.2 |
| 20 | | 4.3 | 7.3 |
| 21 | | 4.5 | 7.5 |
| 22 | | 4.7 | 7.7 |
| 23 | | 4.8 | 7.8 |
| 24 | | 5 | 8.0 |
| 25 | | 5.1 | 8.1 |
| 26 | | 5.3 | 8.3 |
| 27 | | 5.4 | 8.4 |
| 28 | | 5.5 | 8.5 |
| 29 | | 5.7 | 8.7 |
| 30 | | 5.8 | 8.8 |
| 31 | | 5.9 | 8.9 |
| 32 | | 6.1 | 9.1 |
| 33 | | 6.2 | 9.2 |
| 34 | | 6.3 | 9.3 |
| 35 | | 6.5 | 9.5 |
| 36 | | 6.6 | 9.6 |
| 37 | | 6.8 | 9.8 |
| 38 | | 6.9 | 9.9 |
| 39 | | 7.1 | 10.1 |
| 40 | | 7.2 | 10.2 |
| 41 | | 7.4 | 10.4 |
| 42 | | 7.5 | 10.5 |
| 43 | | 7.7 | 10.7 |
| 44 | | 7.8 | 10.8 |
| 45 | | 8 | 11.0 |
| 46 | | 8.1 | 11.1 |
| 47 | | 8.3 | 11.3 |
| 48 | | 8.4 | 11.4 |
| 49 | | 8.5 | 11.5 |
| 50 | | 8.6 | 11.6 |
| 51 | | 8.7 | 11.7 |
| 52 | | 8.8 | 11.8 |
| 53 | | 8.9 | 11.9 |
| 54 | | 9 | 12.0 |
| 55 | | 9 | 12.0 |
| 56 | | 9.1 | 12.1 |
| 57 | | 9.2 | 12.2 |
| 58 | | 9.3 | 12.3 |
| 59 | | 9.4 | 12.4 |
| 60 | | 9.5 | 12.5 |
| 61 | | 9.6 | 12.6 |
| 62 | | 9.7 | 12.7 |
| 63 | | 9.8 | 12.8 |
| 64 | | 9.9 | 12.9 |
| 65 | | 10 | 13.0 |
| 66 | | 10.1 | 13.1 |
| 67 | | 10.1 | 13.1 |
| 68 | | 10.2 | 13.2 |
| 69 | | 10.3 | 13.3 |
| 70 | | 10.4 | 13.4 |
| 71 | | 10.5 | 13.5 |
| 72 | | 10.6 | 13.6 |

a: Age in hours should represent the patient's actual age, i.e., 10.75, which corresponds to the age 10 row (10 hours and 0 minutes to 10 hours and 59 minutes)

Exclusion Criteria—

Patients with any of the following will be excluded from the clinical trial: Elevated direct bilirubin ≥2 mg/dL, or >20% of the total serum bilirubin, alanine aminotransferase (ALT) >2 times the upper limit of normal (ULN) and/or aspartate aminotransferase (AST) >3 times ULN; abnormal renal function defined as creatinine and/or blood urea nitrogen >2 times the ULN; any clinically significant abnormalities on ECG or other screening laboratory evaluation that in the opinion of the investigator makes the patient unsuitable for the clinical trial; apgar score ≤6 at age 5 minutes; an unexplained existing rash or skin erythema; prior exposure to phototherapy; clinical suggestion of neonatal thyroid disease or current uncontrolled thyroid disease in the mother (maternal Hashimoto's disease is not exclusionary); cardio-respiratory distress, defined as a respiratory rate >60 breaths per minute at time of enrollment; any clinically significant abnormal auditory or ophthalmologic findings on screening physical exam; ECG finding of prolonged QTcB during the three screening ECGs: average QTcB >480 ms for neonates on day 1 (0-24 hours) or day 2 (>24-48 hours) of life or an average QTcB >460 ms for neonates on day 3 (>48-72 hours) of life; treatment or need for treatment of the neonate with medications that may prolong the QT interval (see Table 4, certain drugs are known to prolong the QT interval including but not limited to those listed in the table), or family history of Long QT syndrome; known porphyrias or risk factors for porphyrias, including family history; a maternal history of systemic lupus erythematosus; maternal use of phenobarbital within 30 days before, or after delivery if breast-feeding; maternal current drug or alcohol abuse, or maternal history of drug or alcohol abuse that, in the opinion of the Investigator, would not make the patient a suitable candidate for participation in the clinical trial; significant congenital anomalies or infections; risk of requiring surgery or exposure to operating room (OR) lights in the first 2 weeks of life; persistent hypoglycemia unresponsive to medical intervention defined as 3 consecutive readings of blood glucose <40 mg/dl over a 3 hour period; current temperature instability defined as 3 consecutive readings <36° C. and/or >37.5° C. (axillary) over a 3 hour period; use of IVIG or albumin prior to study drug administration; use of photosensitizing drugs or agents (see Table 5, certain drugs may have possible photosensitizing effects including but not limited to those listed in the table); post-delivery treatment with medications that are known or suspected to displace bilirubin from albumin such as, but not limited to, acetaminophen, diazepam, carbamazepine, disopyramide, erythromycin, nitrofurantoin, ibuprofen; exposure to any investigational medications or devices after delivery, or participation in another clinical trial while participating in this trial, or any other concurrent medical condition, which in the opinion of the Investigator makes the patient unsuitable for the clinical trial.

TABLE 4
Drugs Used in Newborns Known
to Prolong the QT Interval

| | |
|---|---|
| Amiodarone | Systemic metronidazole^{b} |
| | (NOTE: |
| | metronidazole is |
| | not a QT prolonging |
| | drug, but it flattens |
| | t-waves) |
| Cisapride | Procainamide |
| Disopyramide | Quinidine |
| Erythromycin^{a} | |

^{a}Erythromycin ointment for eye prophylaxis is permitted.
^{b}The infant will not be eligible for the study if: the mother received metronidazole within two days prior to delivery and the baby is less than 35 hours post last dose of maternal metronidazole administration or the mother began treatment with metronidazole while breast-feeding.

TABLE 5
Photosensitizing Drugs Used in Newborns

| | | | |
|---|---|---|---|
| Amiodarone | Diazoxide | Topical Gentamicin^{a} | Quinidine |
| Captopril | Disopyramide | Hydrochlorothiazide | Ribavirin |
| Cefazolin | Epoetin alfa | Indomethacin | Streptomycin |
| Ceftazidime | Flucytosine | Kanamycin | Verapamil |
| Chlorothiazide | Furosemide | Phenobarbital | |

^{a}Parenteral use of Gentamicin is allowed (only the topical use of Gentamicin is considered photosensitizing and should not be used while the patient is participating in the clinical trial.)

Materials:

For the purposes of this trial, IMP comprises stannsoporfin (Stanate®) and saline solution (placebo), as shown in Tables 6 and 7.

Stannsoporfin drug substance is a magenta-colored powder with a chemical formula of C₃₄H₃₆Cl₂N₄O₄Sn and a molecular weight of 754.30. It is formulated as a solution for IM injection at pH 7.4 to 7.9 and a concentration of 20 mg/mL of tin-mesoporphyrin IX dichloride in a final volume of 1.5±0.2 mL that is contained in a 2.0-mL amber vial.

TABLE 6
Stannsoporfin

| | |
|---|---|
| IMP: | stannsoporfin (Stanate ®) |
| Formulation: | 1.5 ± 0.2 mL of 20 mg/mL |
| | solution of tin-mesoporphyrin |
| | IX dichloride at pH of 7.4-7.9 |
| Dosage Form: | IM injection |
| Treatment Frequency: | Single dose |
| Packaged as: | 2.0-mL amber vial for |
| | IM injection |
| Manufactured by: | InfaCare Pharmaceutical |
| | Corporation |
| Storage Conditions | 20° C. to 25° C. (68° F. to 77° F.), |
| at Clinical Site: | protected from light |
| Lot and Batch : | Will be recorded in the trial |
| Numbers | master file and in the final |
| | clinical trial report |

Saline is a general term referring to a clear-colored sterile solution of sodium chloride in water with a chemical formula of NaCl and a molecular weight of 58.44. It is formulated at a concentration of 0.9% weight-to-volume ratio of sodium chloride.

TABLE 7
Saline solution

| | |
|---|---|
| IMP: | Saline solution |
| Formulation: | Normal saline (0.9%) |
| | weight-to-volume ratio |
| | of sodium chloride |
| Dosage Form: | IM Injection (0.5 mL) |
| Treatment Frequency: | Single dose |
| Storage Conditions at | Room temperature |
| Clinical Site: | 20° C. to 25° C. |
| | (68° F. to 77° F.) |

### Randomization

Once the patient's TSB is at or above the age-specific threshold for phototherapy and all inclusion and no exclusion criteria are met (including laboratory criteria), patients will be immediately randomized to receive a single intramuscular dose of either placebo (saline), 3.0 mg/kg of stannsoporfin or 4.5 mg/kg of stannsoporfin.

Ninety patients will be randomized using a stratified randomization in a 1:1:1 ratio to each of the three treatment arms using a blocked randomization with block sizes of 6. The stratification will be based on either ABO/Rh incompatibility confirmed by blood typing, or G6PD deficiency status. Any patient diagnosed with both ABO incompatibility and G6PD deficiency will be stratified to the G6PD deficiency group.

Assessments and procedures will be performed according to Table 8. A total of approximately 9.45 mL of blood will be taken over a 30-day period (±3 days) for the trial.

TABLE 8
Time and Event Table

| | Assessment | | | | | | | | | | | | | | |
|---|---|---|---|---|---|---|---|---|---|---|---|---|---|---|---|
| | | | TSB at or | | | | | | | | | | | | |
| | | | above the | | | | | | | | | | | | |
| | Enrollment/ | | threshold | | | | | | | | | | | | |
| | Screening | | for PT | Post-Treatment Period | | | | | | | | | | | |
| | Day(s) | | | | | | | | | | | | | | |
| | | | | | | | | | | | | After | | | |
| | | | | | | | | | | | | 48 h | | | |
| | | | | | | | | | | | | until | | | 30 d/ |
| | | | | | | | | | | | | PT is | | | Early |
| | 0 | | | 1-2 d | | | | | | | | discontinued | 7 d | 14 d | Term |
| | | | Time from Dose (hours) | | | | | | | | | | | | |
| | | | | | | | | | | | 48 | | | | |
| | | | | | | | | | | | or | every | | | |
| | | | 0 min | 2 | 6 | 12 | 18 | 24 | 30 | 36 | DC | 24 h | 7 d | 14 d | 30 d |
| | | | | Window | | | | | | | | | | | |
| | | | | ±30 m | ±2 h | | | | | | ±6 h | | ±3 d | ±3 d | ±3 d |
| Informed | X | | | | | | | | | | | | | | |
| consent | | | | | | | | | | | | | | | |
| Inclusion/exclusion | | X | | | | | | | | | | | | | |
| criteria; | | | | | | | | | | | | | | | |
| demographics; | | | | | | | | | | | | | | | |
| med history | | | | | | | | | | | | | | | |
| Concomitant | | X | | X | X | X | | X | | X | X | X | X | | X |
| medications | | | | | | | | | | | | | | | |
| Adverse | | X | | X | X | X | | X | | X | X | X | X | | X |
| events | | | | | | | | | | | | | | | |
| Chemistry | | X | | | | | | | | | X | | X | | X |
| Panel | | | | | | | | | | | | | | | |
| CBC with | | X | | | | X | | | | | X | | X | | X |
| reticulocyte | | | | | | | | | | | | | | | |
| count^{%} | | | | | | | | | | | | | | | |
| TSB | | X | | | X | X | X | X | X | X | X | X every | X | | |
| | | | | | | | | | | | | 12 h | | | |
| DAT | | ------------------------------------X-------- | | | | | | | | | | | | | |
| | | ---------------------------------- | | | | | | | | | | | | | |
| Vital signs | | X | | X | | X | | X | | | X | X | X | | X |
| Examinations: | | | | | | | | | | | | | | | |
| Physical (w/ | | X | | | | | | | | | X | | X | | X |
| routine eye | | | | | | | | | | | | | | | |
| exam) | | | | | | | | | | | | | | | |
| Neurologic | | X | | | | | | | | | X | | | | X |
| Dermatologic | | X | | X | X | X | | X | | X | X | X every | X | | X |
| | | | | | | | | | | | | 12 h | | | |

| Audiology | --------------------------------------------------------X------------------ | | | | |
|---|---|---|---|---|---|
| (ABR, A- | -------------------------------------------- | | | | |
| ABR, BAEP) | | | | | |
| ECGs | X | | X | X | X |
| | (x3) | | | | |
| Randomization | | X | | | |
| (IXRS) | | | | | |
| Study Drug | | X | | | |
| Treatment | | | | | |

| | |
|---|---|
| Phototherapy | X----------------------------------------------------------------------- |
| | ---------------------------------------- |

### Post-Treatment Period

Post treatment assessments occur at 2, 6, 12, 18, 24, 30, 36, 48 hours, and 7 and 30 days post-study drug administration. The assessments and procedures listed in the respective time point columns under Post-Treatment Assessments in the Time and Event Table (Table 8) are to be performed. DAT should be done 12 hours after randomization if not already obtained.

Criteria for discontinuing phototherapy: The criteria for discontinuing phototherapy will be standardized. If a TSB measurement is obtained during phototherapy treatment that is below the age-specific threshold for phototherapy (FIG. 1), phototherapy should be discontinued. In order to check for rebound hyperbilirubinemia, a TSB level is to be obtained between 6 and 12 hours after phototherapy is discontinued (must be done before discharge). If the patient remains in the hospital and continues on phototherapy past 48 hours further assessments and procedures are to be conducted as listed in the Time and Event Table (Table 8).

### Phototherapy (PT)

Initiation of Phototherapy: Once the TSB result has been received, the administration of study drug and initiation of phototherapy should occur as soon as practical. phototherapy should be started within a window of no more than ±30 minutes from IMP administration. The assessments and procedures listed in Table 8 are to be performed.

Phototherapy must be narrow spectrum blue light with a single overhead unit. The height of the phototherapy unit should be adjusted to obtain an irradiance of 30 microwatts/cm2/nm at the level of the patient's abdomen. Irradiance should be measured and adjusted to maintain 30 microwatts/cm2/nm, until phototherapy is discontinued. The recordings are to be documented in the eCRF.

Monitoring during phototherapy: It is essential to protect the patient's eyes with opaque eye patches. These must be checked periodically to ensure complete eye coverage during phototherapy. Use of biliblankets or home phototherapy is not permitted. TSB levels will be done at 6, 12, 18, 24, 30, 36±2 h, and 48 hours±6 h after study drug administration (Table 8). If the patient continues on phototherapy past 48 hours, the TSB will be obtained every 12±6 h.

Note: TSB samples may be obtained for the clinical purposes that may not coincide with the time points for the protocol-mandated TSB sampling. However, TSB samples must still be drawn at all times specified in the Time and Events Table (Table 8).

The criteria for discontinuing phototherapy will be standardized for this study. If a TSB measurement is obtained that is below the age-specific threshold for phototherapy, phototherapy should be discontinued.

Rebound Assessment: Before discharge, to assess for rebound hyperbilirubinemia, a follow-up TSB will be obtained at 6 to 12 hours after discontinuation of phototherapy. If the TSB rebounds to a level at or above the age-specific phototherapy threshold according to the AAP guidelines before the patient is discharged from the hospital, phototherapy should be re-initiated and procedures based on time from dose will be performed according to Time and Events Schedule (Table 8). If a patient was discharged to home and is readmitted for assessment or treatment of hyperbilirubinemia, and has a TSB level at or above the age-specific phototherapy threshold according to the AAP guidelines, phototherapy should be re-initiated.

### Exchange Transfusion (ET)

If a patient has (or is re-admitted for) a TSB level that is at or above the threshold for Exchange Transfusion, the AAP guidelines for the initiation of Exchange Transfusion should be followed (FIG. 2). Assessments in the Time and Events Table (Table 8) should continue to be performed based on time from dose.

### Laboratory Evaluations

Hematology, Clinical Chemistry and Specialty Tests: The clinical site will collect blood samples from patients for analysis. Hematology (Table 9) will be performed at screening, 12, and 48 hours after treatment, and days 7 and 30 or early termination, if applicable (Table 8). If DAT status is unknown or negative, a CBC with reticulocyte should be drawn as soon as possible after enrollment to monitor reticulocyte count for inclusion criteria. Clinical chemistry (Table 9) will be performed at screening, 48 hours and days 7 and 30 or early termination, if applicable (Table 8).

TABLE 9
List of Hematology, Clinical Chemistry and Specialty Tests

| | Clinical Chemistry | | |
|---|---|---|---|
| | | Liver | |
| | Metabolic | Function | Renal |
| Hematology | Tests | Tests | Tests |
| Hemoglobin | Glucose | Total and direct | Blood urea |
| | | serum bilirubin | nitrogen |
| Hematocrit | Total serum | Alkaline | Serum |
| | protein | phosphatase | creatinine |
| Red blood | Albumin | Alanine | |
| cell count | | aminotransferase | |
| Red blood | Calcium | Aspartate | |
| cell indices | | aminotransferase | |
| Reticulocytes | Sodium | Gamma- | |
| | | glutamyl- | |
| | | transferase | |
| White blood | Potassium | | |
| cell count | | | |
| with differential | | | |
| Neutrophils | Chloride | | |
| Lymphocytes | Carbon dioxide | | |
| Monocytes | | | |
| Eosinophils | | | |
| Basophils | | | |
| Mean platelet volume | | | |
| Platelet count* | | | |
| DAT (one time only)** | | | |

*If a patient has a clinically significant laboratory abnormality or a platelet level below 150,000/μl at day 7, the laboratory test should be repeated at day 14.
**DAT should be done at 12 hours after randomization if not already obtained.

Electrocardiogram Methodology: Standard resting 12-lead ECGs will be recorded using a standardized ECG machine that will make interval measures in order to exclude pathology such as prolonged QT. The three screening electrocardiograms will be reviewed by the PI for clinically significant abnormalities and to ensure that no exclusion criteria are met. For patients to be eligible for randomization, the average of 3 QTcB values (from screening ECGs) must not be >480 ms for neonates on day 1 (0-24 hours) or day 2 (>24-48 hours) of life or an average QTcB >460 ms for neonates on day 3 (>48-72 hours) of life.

ECGs will be obtained at screening (3 consecutive ECGs) and single ECGs will be obtained at 2 (corresponding to Tmax), 12 and 48 hours post study drug administration (Table 8). ECGs should be obtained with the patient in a quiet resting state and before any other procedure (e.g., blood draws).

Summary of Statistical Analysis Methods: A statistical analysis plan will detail how the data from this study will be analyzed. The objective of this clinical trial is to assess the safety and efficacy of stannsoporfin in combination with phototherapy in term and near-term neonates. The hypothesis is that treatment with stannsoporfin in combination with phototherapy as opposed to phototherapy only will significantly decrease TSB after drug therapy, and specifically at 48 hours after drug treatment.

Determination of Sample Size: Data from a previous study demonstrated the increase in TSB at 48 hours was higher in placebo (41%) than in the 3.0 and 4.5 mg/kg stannsoporfin patients (15%) receiving phototherapy. Study 204 will have 90% power to detect this difference with a one-sided alpha of 0.025 and N=30 in each of the three treatment arms. This will enable us to detect a difference of 26 percentage points or 2.1 mg/dL at 48 hours.

### Efficacy Endpoints

Primary efficacy endpoint: The primary efficacy endpoint for this study is the percent change in TSB levels from baseline (the baseline TSB is the TSB that qualifies for randomization) at 48 hours post drug treatment.

Secondary efficacy endpoint: The time course of the percent change from baseline in TSB (the baseline TSB is the TSB that qualifies for randomization), total serum bilirubin area under the curve (AUC) above the baseline TSB (0 to 48 hours post-treatment), peak serum bilirubin, incidence of rebound hyperbilirubinemia defined as an increase in TSB above the age-specific threshold for initiating phototherapy following the discontinuation of the initial phototherapy, incidence of readmission to hospital for hyperbilirubinemia due to a TSB at or above the age-specific threshold for phototherapy, duration of clinical requirement for phototherapy defined as the interval between the initiation of phototherapy and the time the bilirubin level crosses below the age-specific threshold for phototherapy, additional analyses of TSB (such as crossing defined threshold values) will be performed.

For all numerically continuous efficacy endpoints, treatment group means will be compared using a full model two-way analysis of covariance (ANCOVA) with a factor for treatment group, a factor for G6PD deficiency, covariate baseline TSB, and an interaction term. The following four pair-wise comparisons will be performed using the appropriate ANCOVA contrast:
stannsoporfin 4.5 mg/kg group vs. placebo
stannsoporfin 3.0 mg/kg group vs. placebo
stannsoporfin 4.5 mg/kg and 3.0 mg/kg vs. placebo
stannsoporfin 3.0 mg/kg vs. stannsoporfin 4.5 mg/kg

For all categorical efficacy endpoints (e.g. exceeding predefined TSB levels, incidence of rebound, readmission rate and exchange transfusion), proportions will be compared using a logistic regression with a factor for treatment group relative to placebo and a factor for G6PD deficiency relative to G6PD deficiency. The same four pair-wise comparisons as stated above will be performed.

### Safety Outcome Measures

Safety outcome measures are: Incidence of adverse events and serious adverse events, changes in vital sign measurements, results of physical exam (PE) including eye and hearing assessment, results of neurologic exam, ECG assessments, clinical laboratory tests including hematology, serum chemistries, liver function and renal function tests. Adverse events and SAEs will be summarized for each treatment group and overall for both stannsoporfin treatment groups with the proportion of patients reporting each event. Actual values and change from baseline in vital signs, physical and neurologic exam and laboratory test parameters will be summarized for each treatment group and overall for both stannsoporfin treatment groups with descriptive statistics at each assessment obtained. Changes in ECG findings, ophthalmologic and audiology assessments will be summarized for each treatment group and overall for both stannsoporfin treatment groups with descriptive statistics at each assessment obtained, including changes from baseline to Day 30 (early term). Efficacy and safety parameters will also be analyzed according to DAT status.

### Results

The results from a Phase 2b multicenter, double blind, randomized, placebo controlled parallel group safety and efficacy trial of two doses of stannsoporfin administered as a single intramuscular (IM) injection in combination with phototherapy (PT) in term and near-term infants, all with an ABO/Rh incompatibility were highly statistically significant and positive. Ninety-one neonates (aged 24 hours of age, range 7 to 54 hours), all at medium to high risk of severe hyperbilirubinemia, all with an ABO/Rh incompatibility, birth weights of 2580 to 4930 grams, and gestational age of 35 to 42 weeks, were randomized to one of three treatment arms: placebo (saline), 3.0 mg/kg stannsoporfin, or 4.5 mg/kg of stannsoporfin. All were included in the intent-to-treat-primary efficacy and safety analyses.

Stannsoporfin showed a highly significant (P<0.0001) positive effect for both the high dose (4.5 mg/kg) and the low dose (3.0 mg/kg) on total serum bilirubin compared with phototherapy and placebo in its primary efficacy endpoint, percentage change in total serum bilirubin (TSB) at 48 hours after drug administration. The primary efficacy endpoint was based on the TSB at 48 hours. The primary efficacy endpoint was the LS Mean Difference from placebo in the TSB percentage change from baseline at 48 hours. The analysis was based on the last observation carried forward (LOCF), when the 48 hour value was not available (window 40 to ≤54 hours from time of dose). Results are summarized in Table 10. All infants received phototherapy from the time of study dosing.

TABLE 10
Primary Efficacy Analysis

| | Stannsoporfin | Stannsoporfin | Stannsoporfin | |
|---|---|---|---|---|
| Parameter | Combined | 3.0 mg/kg | 4.5 mg/kg | Placebo |
| LS Mean | (N = 61) | (N = 30) | (N = 31) | (N = 30) |
| LS Mean | −7.4 | −9.5 | −5.5 | 22.2 |
| Percentage | (−13.56, −1.29) | (−18.23, −0.69) | (−14.09, 3.17) | (13.46, 31.00) |
| Change in TSB | | | | |
| at 48 hrs (95% | | | | |
| CI) | | | | |
| LS Mean | −29.69 | −31.69 | −27.69 | |
| Difference vs | (−40.41, −18.97) | (−44.10, −19.28) | (−39.99, −15.38) | |
| Placebo in TSB | P < 0.0001 | P < 0.0001 | P < 0.0001 | |
| at 48 hrs (95% | | | | |
| CI), P-value | | | | |

Notably, stannsoporfin showed a statistically significant positive effect for both the high dose (4.5 mg/kg) and the low dose (3.0 mg/kg) on total serum bilirubin compared with phototherapy and placebo in its primary efficacy endpoint, percentage change in total serum bilirubin (TSB) at 48 hours after drug administration and a highly statistically significant (P<0.0001) positive effect for both the high dose (4.5 mg/kg) and the low dose (3.0 mg/kg) on total serum bilirubin compared with phototherapy and placebo in its primary efficacy endpoint, percentage change in total serum bilirubin (TSB) at 24 hours after drug administration.

In addition, there were three key secondary efficacy endpoints, which were pre-specified and strictly tested in an a priori testing order. For the high dose, the first two of the three key secondary endpoints were statistically significant, but not for the low dose treatment arm. See Table 11.

TABLE 11
Secondary Efficacy Analysis

| Parameter | Stannsoporfin | | Stannsoporfin | | Stannsoporfin | | | |
|---|---|---|---|---|---|---|---|---|
| (In strict | Combined | | 3.0 mg/kg | | 4.5 mg/kg | | Placebo | |
| testing Order) | (N = 61) | | (N = 30) | | (N = 31) | | (N = 30) | |
| 1. Time at which | 11.6 hrs | | 11.8 hrs | | 10.6 hrs | | 20.9 | |
| TSB crosses at or | (8.7, 16.1) | | (8.2, 21.5) | | (8.1, 16.4) | | (9.2, 26.5) | |
| below the PT | P = 0.021 | | P = 0.189 | | P = 0.003 | | | |
| threshold Median, | | | | | | | | |
| (95% CI), | | | | | | | | |
| P-value vs Placebo | | | | | | | | |
| 2. PT Failure N (%) | 4/61 | (7%) | 3/30 | (10%) | 1/31 | (3%) | 8/30 | (27%) |
| Odds Ratio | 0.20 | | 0.31 | | 0.09 | | | |
| vs Placebo | (0.039, 0.824) | | (0.048, 1.498) | | (0.002, 0.793) | | | |
| (95% CI) | P = 0.023 | | P = 0.181 | | P = 0.023 | | | |
| P-value vs Placebo | | | | | | | | |
| 3. Rebound | 1/61 | (2%) | 0/30 | (9%) | 1/31 | (3%) | 3/30 | (10%) |

| | | | |
|---|---|---|---|
| Hyperbilirubinemia | 0.206 | 0.237 | 0.587 |
| N (%) | | | |
| P-value vs Placebo | | | |

PT Failure is defined as a binary variable on each of four components: exchange transfusion, IVIG drug used, hospital readmission for hyperbilirubinemia and re-started PT. If any component is positive the PT failure is considered to have occurred.
Rebound Hyperbilirubinemia is defined as an increase in TSB at or above the age-specific threshold for initiating PT following the discontinuation of the initial PT therapy.

Safety: There were no infant deaths and no infant discontinued the study due to an adverse event. Overall 52% of infants had a treatment-emergent adverse event (TEAE). TEAEs occurred in 59% of Stannsoporfin treated infants vs 52% among placebo. Serious TEAEs occurred in 15% of Stannsoporfin treated infants vs 20% of placebo. TEAEs related to phototherapy (eg skin rash, erythema) occurred in 16% of Stannsoporfin treated infants vs 10% of placebo. There were no safety signals with either dose of Stannsoporfin.

Although the methods disclosed herein have been described in considerable detail with reference to certain preferred embodiments thereof, other versions are possible. Therefore, the spirit and scope of the appended claims should not be limited to the description and the preferred versions contained within this specification.

1. A method of decreasing bilirubin levels in an infant in need thereof, the method comprising:
initiating phototherapy in the infant and substantially simultaneously with the initiation of phototherapy, administering to the infant a therapeutic amount of stannsoporfin.

2. The method of claim 1, wherein the administering of the therapeutic amount of stannsoporfin occurs no more than 60 minutes apart from the initiation of phototherapy.

3. (canceled)

4. The method of claim 1, wherein the administering of the therapeutic amount of stannsoporfin occurs no more than 60 minutes before the initiation of phototherapy.

5. (canceled)

6. The method of claim 1, wherein the administering of the therapeutic amount of stannsoporfin occurs no more than 60 minutes after the initiation of the phototherapy.

7. (canceled)

8. The method of claim 1, wherein the therapeutic amount of stannsoporfin is selected from the group consisting of 1.5 mg/kg, 3.0 mg/kg and 4.5 mg/kg.

9. The method of claim 1, wherein prior to initiating phototherapy, the infant has a total serum bilirubin level at or above the age-specific threshold for initiating phototherapy per the AAP guidelines.

10. The method of claim 1, wherein phototherapy is stopped when the total serum bilirubin level crosses below the age-specific threshold for initiating phototherapy per the AAP guidelines.

11. The method of claim 1, wherein the infant is direct antiglobulin test (DAT) negative with a reticulocyte count greater than 6%.

12. The method of claim 1, wherein prior to initiating phototherapy, the infant is about twenty-four hours old or less.

13. (canceled)

14. (canceled)

15. The method of claim 1, wherein prior to initiating phototherapy, the infant has a total serum bilirubin level within 3 mg/dL the threshold for phototherapy initiation of the AAP guidelines.

16. The method of claim 1, wherein the infant is at increased risk for hyperbilirubinemia.

17. The method of claim 16, wherein the increased risk is due to hemolytic disease, ABO incompatibility, Rh incompatibility, or G6PD deficiency.

18. The method of claim 16, wherein the increased risk is measured as a rate of rise of bilirubin greater or equal to 0.2 mg/dL/hour.

19. A method of treating hyperbilirubinemia in an infant in need thereof, the method comprising:
initiating phototherapy in the infant and substantially simultaneously with the initiation of phototherapy, administering to the infant a therapeutic amount of stannsoporfin, wherein prior to initiating phototherapy, the infant is 24 hours old or less.

20. The method of claim 19, wherein the administering of the therapeutic amount of stannsoporfin occurs no more than 60 minutes apart from the initiation of phototherapy.

21-25. (canceled)

26. The method of claim 19, wherein the therapeutic amount of stannsoporfin is selected from the group consisting of 1.5 mg/kg, 3.0 mg/kg and 4.5 mg/kg.

27. The method of claim 19, wherein prior to imitating phototherapy, the infant has a total serum bilirubin level at or above the age-specific threshold for initiating phototherapy per the AAP guidelines.

28. The method of claim 19, wherein phototherapy is stopped when the total serum bilirubin level crosses below the age-specific threshold for initiating phototherapy per the AAP guidelines.

29. The method of claim 19, wherein the infant is direct antiglobulin test (DAT) negative with a reticulocyte count greater than 6%.

30-32. (canceled)

33. The method of claim 19, wherein prior to initiating phototherapy, the infant has a total serum bilirubin level within 3 mg/dL the threshold for phototherapy initiation of the AAP guidelines.
